(19) United States
(12) Patent Application Publication
(10) Pub. No.: US 2005/0036977 A1
(43) Pub. Date: Feb. 17, 2005

(54) Taste-masked resinate and preparation thereof

(76) Inventors: Dilip Gole, Plainsboro, NJ (US); Traci Savall, San Diego, CA (US); Lyou-fu Ma, Ann Arbor, MI (US); Dale Greenwood, Fishers, IN (US); Paul K. Wilkinson, Titusville, NJ (US); John Desmond Davies, Princeton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/638,822

(22) Filed: Aug. 11, 2003

(51) Int. Cl. A61L 9/01; A61L 9/00

(52) U.S. Cl. 424/76.1

(57) ABSTRACT

The present invention is directed to a taste-masked resinate that contains a water-insoluble active substance complexed to an ion-exchange resin in a taste-masking effective amount. The taste-masked resinate is useful in the manufacture of a dosage form such as a rapid-disintegrating tablet, a rapid-disintegrating film, an effervescent tablet, a chewable tablet, a chewing gum, a suspension, a sprinkle granule, a powder for reconstitution in a suspension and the like and a method for the preparation thereof.

### FIELD OF THE INVENTION

The present invention is directed to a taste-masked resinate, a method for the preparation thereof and a method for the use thereof. The taste-masked resinate comprises a water-insoluble bitter-tasting, pharmaceutically active substance and an ion-exchange resin complexed in a taste-masking effective amount. The taste-masked resinate may be employed in the manufacture of pharmaceutical compositions, including dosage forms such as rapid-disintegrating tablets, rapid-disintegrating films, effervescent tablets, chewable tablets, chewing gum, suspensions, sprinkle granules and powder for reconstitution in suspension.

### BACKGROUND OF THE INVENTION

Many pharmaceutically active substances are presented for oral administration in tablet, pill, capsule or powder dosage form. The dosage form is swallowed so that the pharmaceutically active substance can be absorbed via the gastro-intestinal tract. For pediatric and geriatric patients and for patients having a central nervous system disorder, swallowing such dosage forms may be difficult or impossible. Approximately 35-50% of the population finds it difficult to swallow an oral dosage form.

Because they do not require the use of water or chewing to administer, orally disintegrating dosage forms, also known, for example, as “fast-melt”, “rapid-melt”, “rapid-disintegrating” or “quick-disintegrating” dosage forms (referred to in the European Pharmacopoeia as “orodispersable tablets” or generally as “orally disintegrating tablets”) are easy to use, convenient and patient friendly. The term “fast-melt” generally refers to a tablet composition wherein an active substance or drug is distributed or dispersed in a carrier matrix which disintegrates in the oral cavity upon administration; and, upon swallowing and subsequent absorption, releases the drug for entry to the gastrointestinal tract. The term “oral cavity” includes the entire interior of the mouth, including not only the buccal cavity (that part of the oral cavity anterior to the teeth and gums) but also the sublingual and supralingual spaces.

Typical fast-melt tablets, i.e., those that include a carrier matrix and an active ingredient, begin to lose their rapid-disintegration characteristics as the relative amount of active substance in the tablet increases. The high amount of active substance required for therapeutic effectiveness may be a limiting factor in whether a typical fast-melt tablet can be used. As a result, several tablets having a low drug loading may have to be administered, possibly resulting in patient inconvenience and decreased compliance. Moreover, if the fast-melt carrier matrix disintegrates in the oral cavity, the pharmaceutically active substance, which may have a bitter taste, is released into the oral cavity.

Accordingly, there remains a need for a rapid-disintegrating oral dosage form having a therapeutically effective dose of a taste-masked bitter tasting pharmaceutically active substance.

Rapidly disintegrating solid oral dosage forms are known. For example, U.S. Pat. No. 5,607,697, entitled “Taste-masking Microparticles for Oral Dosage Forms”, discloses a compressed tablet consisting of taste-masked coated microparticles that disintegrate in the mouth. The microparticles include a sweetener such as mannitol, sorbitol and artificial sweetener. U.S. Pat. No. 5,871,781 entitled “Apparatus for Making Rapidly Dissolving Dosage Units”, discloses comestible units wherein the units are capable of dissolving within several seconds. U.S. Pat. No. 5,869,098 entitled “Fast-Dissolving Comestible Units Formed Under High-Speed/High-Pressure Conditions”, discloses fast-dissolving tablets comprising substantially amorphous sucrose, sorbitol, or xylitol or mixtures thereof. U.S. Pat. Nos. 5,866,163, 5,851,553, and 5,622,719, all entitled “Process and Apparatus for Making Rapidly Dissolving Dosage Units and Product Therefrom”, disclose methods for producing fast-dissolving tablets that contain an active ingredient and a crystalline structure. U.S. Pat. No. 5,567,439 entitled “Delivery of Controlled-Release Systems”, discloses a quick dissolve tablet that can be employed for delivery in a controlled release system. U.S. Pat. No. 5,587,172 entitled “Process for Forming Quickly Dispersing Comestible Unit and Product Therefrom”, discloses the use of a flowable, compactable micro-particulate to form a tablet. U.S. Pat. No. 5,464,632 entitled “Rapidly Disintegratable Multiparticular Tablet”, discloses a fast-melt tablet that contains the active substance in the form of coated or non-coated microcrystals or microgranules. U.S. Pat. No. 4,642,903 entitled “Freeze-Dried Foam Dosage Form”, discloses a fast-melt dosage formulation that contains an active ingredient dispersed in a freeze dried foam. U.S. Pat. No. 5,631,023 entitled “Method for Making Freeze Dried Drug Dosage Forms”, discloses freeze-dried drug dosage forms made by adding xanthan gum to a suspension of gelatin and active agent.

U.S. Pat. No. 5,738,875 entitled “Process for Preparing Solid Pharmaceutical Dosage Forms”, discloses a process for preparing an oral solid rapidly disintegrating dosage form comprising forming a solution or a suspension in an aqueous medium of an uncoated and uncomplexed form of a pharmaceutically active substance which is present in its free base form, said free base form being less soluble in water and more palatable than the corresponding salt form with the unacceptable taste, together with a water-soluble or water-dispersible carrier material and a compound which converts the salt form of the pharmaceutically active substance into its free base form. The patent discloses that the carrier may be gelatin and that the salt may be converted to its free base form using sodium hydrogen carbonate buffer.

U.S. Pat. No. 5,827,541 entitled “Process for Preparing Solid Pharmaceutical Dosage Forms of Hydrophobic Substances”, discloses a solid pharmaceutical dosage form of hydrophobic substances formed by freeze-drying a dispersion containing a hydrophobic active ingredient and a surfactant in a non-aqueous phase and a carrier material in an aqueous phase. The patent discloses that the carrier may be gelatin.

U.S. Pat. No. 5,976,577 entitled “Process for Preparing Fast Dispersing Solid Oral Dosage Form”, discloses a freeze-dried dosage form that contains the active substance uncoated or coated with a water-insoluble polymer or lipid material. The patent discloses that the polymer may be cellulose or gelatin.

U.S. Pat. No. 5,466,464 entitled “Intrabuccally Disintegrating Preparation and Production Thereof”, discloses a solid preparation composed of an active ingredient and a sugar comprising lactose and/or mannitol and agar. U.S. Pat. No. 5,576,014 entitled “Intrabuccally Dissolving Compressed Moldings and Production Process Thereof”, discloses intrabuccally dissolving compressed moldings comprising a saccharide. U.S. Pat. No. 6,465,009 entitled “Water Soluble Polymer-Based Rapidly Dissolving Tablets and Production Processes Thereof” discloses a tablet comprising a water soluble polyvinylpyrrolidone, at least one pharmaceutical-active substance and a saccharide.

U.S. Pats. Nos. 5,639,475 and 5,709,886 entitled “Effervescent Microcapsules”, disclose a rapidly dissolving effervescent composition having a mixture of sodium bicarbonate, citric acid and ethylcellulose. U.S. Pats. Nos. 5,807,578 and 5,807,577 entitled “Fast-Melt Tablet and Method of Making Same”, disclose a fast-melting tablet comprising an active ingredient, an effervescent couple consisting of an effervescence base and an effervescence acid, a starch and a tablet lubricant.

U.S. Pat. No. 5,112,616 and U.S. Pat. No. 5,073,374 both entitled “Fast Dissolving Buccal Tablet”, disclose a fast dissolving tablet including the active ingredient, a lubricant and a water soluble sugar, such as sorbitol. U.S. Pat. No. 4,616,047 entitled “Galenic Form for Oral Administration and Its Method of Preparation by Lyophilization of an Oil-in-Water Emulsion”, discloses a porous dosage form made by pressurized lyophilization of an oil-in-water emulsion. U.S. Pat. No. 5,720,974 entitled “Fast Dissolving Tablet and Its Production”, discloses a method of making a fast dissolving tablet in which an active agent and a soluble carbohydrate which has been barely-moistened are compression molded into a tablet followed by drying. U.S. Pat. No. 6,316,029 entitled “Rapidly Disintegrating Solid Oral Dosage Form”, discloses a rapidly disintegrating solid oral dosage form of a poorly soluble active ingredient and at least one pharmaceutically acceptable water-soluble or water-dispersible excipient. The patent discloses that the excipient may be a sugar, a starch or a natural polymer.

U.S. Pat. No. 5,807,576 entitled “Rapidly Dissolving Tablet”, discloses rapidly dissolving tablets having two polypeptide (or gelatin) components and a bulking agent. U.S. Pat. No. 5,595,761 entitled “Particulate Support Matrix for Making a Rapidly Dissolving Tablet”, discloses a rapidly disintegrating particulate support matrix for making a solid tablet form comprising a first polymeric component (such as a polypeptide, preferably a non-hydrolyzed gelatin) and a second polymeric component (a different polypeptide such as a hydrolyzed gelatin and a bulking agent) to which a pharmaceutical active (such as an antihistamine, decongestant or antibiotic) is added. U.S. Pat. No. 5,587,180 entitled “Process for Making a Particulate Support Matrix for Making a Rapidly Dissolving Tablet”, U.S. Pat. No. 5,635,210 entitled “Method of Making a Rapidly Dissolving Tablet”, U.S. Pat. No. 5,776,491 entitled “Rapidly Dissolving Dosage Form”, U.S. Pat. No. 6,177,104 entitled “Particulate Support Matrix for Making a Rapidly Dissolving Dosage Form” and U.S. Pat. No. 6,207,199 entitled “Process for Making a Particulate Support Matrix for Making a Rapidly Dissolving Dosage Form” disclose a rapidly disintegrating particulate support matrix and a dosage form made therefrom, wherein a porous particulate powder matrix comprises at least two polymeric components having different solubilities. U.S. Pat. No. 6,066,337 entitled “Method for Producing a Rapidly Dissolving Dosage Form”, discloses a rapidly disintegrating dosage form comprising a particulate support matrix of a support agent that includes a non-hydrolyzed gelatin component and a hydrolyzed gelatin component. U.S. Pat. No. 5,648,093 and U.S. Pat. No. 5,558,880 both entitled “Pharmaceutical and Other Dosage Forms”, disclose a fast dissolving, solid dosage form defined by a matrix containing gelatin, pectin and/or soy fiber protein and one or more amino acids having from about 2 to 12 carbon atoms. U.S. Pat. No. 5,330,764 entitled “Methods of Preparing Bulk Delivery Matrices by Solid-State Dissolution”, U.S. Pat. No. 5,330,763 entitled “Delivery Matrices Prepared by Solid-State Dissolution” and U.S. Pat. No. 5,215,756 entitled “Preparation of Pharmaceutical and Other Matrix Systems by Solid-State Dissolution”, disclose a method for preparing a matrix system by solidifying a matrix composition dissolved or dispersed in a first solvent, then adding a second solvent that is substantially miscible with the first solvent at a temperature lower than the solidification point of the first solvent, wherein the solidified matrix is substantially insoluble in the second solvent and whereby the first solvent is substantially removed resulting in a matrix.

U.S. Pat. No. 5,071,646 entitled “Pharmaceutical Ion-exchange Resin Composition”, discloses a resin composition comprising a granulated ion-exchange resin, a pharmacologically active ingredient bound thereto with a sugar or sugar alcohol, and a sufficient amount of water, alcohol or aqueous alcohol to facilitate granulation. The patent discloses that the ratio of active agent to ion-exchange resin may vary between about 1:3 to 2:1. U.S. Pat. No. 5,188,825 discloses freeze-dried dosage forms prepared by bonding or complexing a water-soluble active agent to or with an ion exchange resin to form a substantially water insoluble complex. The patent discloses that the ratio of active agent to ion-exchange resin may vary between about 10:1 to 1:5.

While forming a resinate is known, none of the references discussed above discloses or suggests the taste-masked resinate of the invention that comprises a water-insoluble, bitter-tasting pharmaceutically active substance and an ion-exchange resin complexed in a taste-masking effective amount.

### SUMMARY OF THE INVENTION

The present invention provides a taste-masked resinate comprising a therapeutically effective amount of a water-insoluble, bitter-tasting pharmaceutically active substance complexed in a taste-masking effective amount with an ion-exchange resin.

The taste-masked resinate may comprise a water-insoluble base of an active substance and a cationic ion-exchange resin.

The ratio of water-insoluble active substance to ion-exchange resin is such that the equilibrium concentration of the active substance and the equilibrium concentration of the ion-exchange resin are balanced so that the water-insoluble active substance is complexed with the ion-exchange resin in a taste-masking effective amount, namely in a ratio of water-insoluble active substance to ion-exchange resin between about 1:4 to about 1:12.

The water-insoluble active substance may be complexed with the ion-exchange resin in a medium such as water or a buffered aqueous solution. The medium is optionally balanced by a neutralizing agent to favor the water-insoluble active substance complexing with the ion-exchange resin in a taste-masking effective amount. The neutralizing agent may be used to ensure that the pH is in a range from about pH 5 to about pH 10.

The invention provides a method for preparing a taste-masked resinate comprising adding a specified amount of a water-insoluble active substance to a specified amount of an ion-exchange resin such that a ratio of water-insoluble active substance complexed to ion-exchange resin is between about 1:4 to about 1:12.

The taste-masked resinate may be employed in the manufacture of a pharmaceutical composition including dosage forms such as rapid-disintegrating tablets, rapid-disintegrating films, effervescent tablets, chewable tablets, chewing gum, suspensions, sprinkle granules or powder for reconstitution in suspension. The invention thus further provides dosage forms containing the taste-masked resinate.

The invention provides a method for preparing a taste-masked pharmaceutical composition comprising the steps of:
forming a taste-masked resinate of the invention in an aqueous medium, wherein the taste-masked resinate comprises a therapeutically effective amount of a water-insoluble, bitter-tasting pharmaceutically active substance complexed with an ion-exchange resin, wherein the aqueous medium comprises water and optionally one or more of the following: neutralizing agents, sweetening agents, flavoring agents, coloring agents, anti-foaming agents and the like or mixtures thereof;
forming a plurality of taste-masked resinate dosage units; and
removing the water from the dosage units by drying by, for example, lyophilization, centrifugation, spray-drying, evaporation with or without a heat source, filtration with or without a vacuum or combinations thereof.

The invention further includes a method wherein a therapeutically effective amount of a water-insoluble base form of an active substance and a cationic ion-exchange resin are maintained in a ratio whereby the active substance complexes with the resin in a taste-masking effective amount, namely in a ratio of water-insoluble active substance to ion-exchange resin between about 1:4 to about 1:12.

In a preferred embodiment, the invention includes a method for preparing a rapid-disintegrating, taste-masked tablet that comprises the steps of:
forming a taste-masked resinate of the invention in an aqueous medium, wherein the taste-masked resinate comprises a therapeutically effective amount of a water-insoluble, bitter-tasting pharmaceutically active substance complexed with an ion-exchange resin, wherein the aqueous medium comprises water and optionally one or more of the following: carrier materials, structure-imparting water-soluble excipients, thickening agents, neutralizing agents, sweetening agents, flavoring agents, coloring agents, anti-foaming agents and the like or mixtures thereof;
optionally balancing the medium pH by adding a neutralizing agent, whereby an equilibrium concentration of the ion-exchange resin and the water-insoluble active substance is shifted to favor substantially complexing the water-insoluble active substance with the ion-exchange resin, whereby a taste-masking effective amount of the active substance is maintained;
imparting viscosity to the medium by, for example, adding a thickening agent to the medium or homogenizing the medium, wherein the thickening agent is selected from a carbomer, a natural gum or mixtures thereof;
forming a plurality of taste-masked resinate dosage units; and
removing the water from the dosage units by drying by, for example, lyophilization, centrifugation, spray-drying, evaporation with or without a heat source, filtration with or without a vacuum or combinations thereof.

A preferred method of the present invention comprises the steps of:
mixing the ingredients to form a taste-masked resinate of the invention in a suspension;
filling a plurality of divided dosage units with the suspension;
drying the suspension in the dosage units using a drying means to form a plurality of resinate dosage units. The resinate dosage units may then be employed to manufacture a pharmaceutical composition such as described above.

### DETAILED DESCRIPTION OF THE INVENTION

The present invention is directed to a taste-masked resinate comprising a therapeutically effective amount of a water-insoluble, bitter-tasting pharmaceutically active substance complexed in a taste-masking effective amount with a water-insoluble ion-exchange resin.

The taste-masked resinate may be employed in the manufacture of a pharmaceutical composition including dosage forms selected from rapid-disintegrating tablets, rapid-disintegrating film; effervescent tablets, chewable tablets, chewing gum, suspensions, sprinkle granules or powder for reconstitution in suspension. A preferred embodiment is a rapid-disintegrating tablet or a suspension.

Embodiments of the present invention include a rapid-disintegrating tablet having a disintegration time selected from a time in a range of less than about 60 seconds, a time in a range of less than about 30 seconds, a time in a range of less than about 20 seconds, a time in a range of less than about 15 seconds, a time in a range of less than about 10 seconds, a time in a range of less than about 5 seconds or a time in a range of less than about 3 seconds.

Embodiments of the present invention include a rapid-disintegrating tablet having a disintegration time selected from a time in a range of from about 1 second to about 60 seconds, a time in a range of from about 1 second to about 30 seconds, a time in a range of from about 1 second to about 20 seconds, a time in a range of from about 1 second to about 15 seconds, a time in a range of from about 1 second to about 10 seconds, a time in a range of from about 1 second to about 5 seconds or a time in a range of from about 1 second to about 3 seconds.

Embodiments of the present invention include a rapid-disintegrating tablet having a disintegration time selected from a time in a range of from about 3 seconds to about 60 seconds, a time in a range of from about 5 seconds to about 60 seconds, a time in a range of from about 5 seconds to about 30 seconds, a time in a range of from about 5 seconds to about 20 seconds, a time in a range of from about 5 seconds to about 15 seconds, a time in a range of from about 5 seconds to about 10 seconds or a time in a range of about 5 seconds.

The invention provides a rapid-disintegrating, taste-masked oral dosage form comprising a therapeutically effective amount of a water-insoluble base form of a bitter-tasting active substance and an ion-exchange resin. The invention can be practiced with a wide variety of active substances and ion-exchange resins.

The bitter-tasting active substance may be selected from a variety of known drug classes including, but not limited to, an analgesic agent, antiinflammatory agent, antipyretic agent, anthelmintic agent, cardiovascular agent; antianginal agent, anti-arrhythmic agent, antistroke agent, antihypertensive agent, cardiac inotropic agent, antithrombotic agent, coronary dilator or vasodilator or haemostatic agent, peripheral vasodilator agent, erythropoietic mimetic or agent, hyperglycemic agent, anticoagulant, antidiabetic agent, beta-blocking agent, blood product or substitute, antiasthmatic or bronchodilator agent, cough suppressant (expectorants and mucolytics) or respiratory stimulant agent, antipsychotic or antidepressant or cognition activator or psychotropic agent, anxiolytic sedative agent (hypnotics and neuroleptics) or tranquilizer, cerebral dilator agent, vertigo agent, dopaminergic agent (antiparkinsonian), antiepileptic or anticonvulsant or antispasmodic or muscle relaxant or neuromuscular agent, antimigraine or anticluster headache agent, immunosuppressant or antihistamine or antiallergic agent, antimuscarinic agent, antibiotic or antiviral agent, antiinfective or antimycobacterial or fungicidal or antiparasitic agent, wound healing agent, chemotherapeutic or antineoplastic agent, antithyroid or thyroid agent, parathyroid calcitonin or biphosphonate, beta-adrenoceptor blocking agent, gastrointestinal or antinauseant or antidiarrheal or diuretic agent, chelating agent, immunological agent, antilipid agent or lipid regulating agent, growth regulating agent, anticholesterolemic or cholecystekinin or prostaglandin regulating agent, corticosteroid or anabolic agent, renin inhibitor agent, sex hormone mimetic or hormone replacement agent, contraceptive or fertility agent, uterine relaxant agent, stimulant (e.g. appetite stimulant) or anoretic (e.g. appetite suppressant) agent, parasympathomimetic or sympathomimetic agent and the like. A description of these drug classes and a listing of species within each class can be found in Martindale, The Extra Pharmacopoeia, Twenty-ninth Edition (The Pharmaceutical Press, London, 1989). The bitter-tasting water-insoluble active substance, which can be prepared by techniques known in the art, is present in any amount sufficient to elicit a therapeutic effect and, where applicable, is present in the form of a substantially pure optical enantiomer or as a mixture, racemic or otherwise, of enantiomers.

Embodiments of the present invention include a bitter-tasting water-insoluble antipsychotic agent as the active substance selected from risperidone, paliperidone and analogs and derivatives thereof, including analogs and derivatives disclosed in U.S. Pats. Nos. 4,084,663 and 5,158,952, the entire contents of which are incorporated herein by reference. Other embodiments include an active substance selected from an ester, enantiomer, polymorph or amorphous form of risperidone, paliperidone or of analogs or derivatives thereof. In a particular embodiment of the present invention, the bitter-tasting active substance is risperidone selected from an ester, enantiomer, polymorph or amorphous form of risperidone. In a more particular embodiment, the form of risperidone is a water-insoluble base form. Other particular embodiments include risperidone selected from a polymorph or amorphous form of risperidone. In a more particular embodiment, risperidone is selected from a thermodynamically stable polymorph or amorphous form of risperidone.

The taste-masked resinate of the invention meets the present needs of treating physicians by overcoming the disadvantages of previous formulations. Many psychiatrists have expressed a desire to prescribe risperidone in a taste-masked, rapid-disintegrating tablet or in a taste-masked liquid suspension to minimize the likelihood that their patients will cheek and spit out the then used film-coated risperidone tablets. Others have noted that taste-masking with sweeteners or established flavor systems to date have not adequately masked the bitter taste of risperidone.

The taste-masked risperidone resinate may be employed in the manufacture of a rapid-disintegrating tablet form or of a liquid suspension form, each form of which provides advantages. The rapid-disintegrating tablet makes it difficult for the patient to cheek the tablet and spit it out. It is less likely that the risperidone resinate, rapid-disintegrating tablet or liquid suspension would be rejected because of taste, thus tending to greatly increase patient compliance.

The method of the invention, which involves the formation of a resinate by adding a specified amount of a water-insoluble active substance and a specified amount of an ion-exchange resin, provides further advantages in that it involves a significantly simplified manufacturing process, wherein a number of manufacturing steps art are avoided. For example, the process steps of solubilizing an active substance, converting or otherwise changing a salt form of an active substance to a free acid or free base form (as in U.S. Pat. No. 5,738,875), forming a hydrate of an active substance or changing an active substance to a polymorphic form thereof and the like to make the active more palatable can be avoided. Other process steps may be avoided, such as washing, filtering and drying the resinate, screening the resinate to remove any agglomerates, fractionating the resinate to obtain a particular particle size, assaying the resinate before it can be incorporated into the carrier material and the like. Some other steps that may also be avoided include adjusting the sugar or sugar alcohol concentration of the aqueous medium, adjusting the active substance particle size (as in U.S. Pat. No. 6,316,029), fractionating the active substance particle size, pre-cooling an aqueous medium containing a resinate (as in U.S. Pat. No. 5,976,577), separately forming the resinate by physically isolating the complexing active substance and the like.

The taste-masked resinate of the invention displays surprising characteristics. A particularly advantageous characteristic is that the resinate is useful in a variety of dosage forms.

A pharmaceutical composition of the invention may be prepared using the following: a water-insoluble active substance and an ion-exchange resin; and, optionally, one or more of the following: an aqueous medium; a solvent; a carrier material; a surface stabilizer; structure-imparting water-soluable or water-dispersible excipient; thickening agent; filling agent; lubricating agent; suspending agent; neutralizing agent; sweetening agent; flavoring agent; coloring agent; anti-foaming agent; preservative; diluent; and effervescent agent.

The cationic (strongly acidic) ion-exchange resin Amberlite Resin Grade IRP-69 (a trade name of Rohm and Haas Company) uses the sodium ion as the exchange cation. The use of Amberlite Resin IRP-69 disadvantageously requires that a weakly basic active substance such as risperidone be converted into a water soluble acid salt, such as the hydrochloric acid salt. Because the exchange ion sodium irreversibly binds with the chloride ion of the hydrochloric acid salt form of the weakly basic risperidone active substance, the strongly acidic Amberlite resin IRP-69 also disadvantageously forms a strong bond with the weakly basic active substance, thus releasing none of the active substance in the strongly acidic environment of the stomach.

The cationic (weakly acidic) ion-exchange resin Amberlite Resin Grade IRP-88 (a trade name of the Rohm and Haas Company) uses the potassium ion as the exchange ion. The use of Amberlite IRP-88 also disadvantageously requires that a weakly basic active substance be converted into a water-soluble acid salt. The weakly acidic Amberlite Resin IRP-88, though, readily binds with and reversibly releases a weakly basic active substance under acidic conditions. However, the resinate formed using Amberlite Resin IRP-88 disadvantageously must be washed to remove the potassium ions released during formation of the complex. Washing is also necessary to eliminate the salty taste associated with potassium ions. Furthermore, the resinate formed in the aqueous medium is difficult to freeze dry due to the presence of potassium ions which cause eutectic crystal formation in the resinate during the freezing process.

The cationic (weakly acidic) ion-exchange resin Amberlite Resin Grade IRP-64 (a trade name of the Rohm and Haas Company for a brand of polacrilex resin; hereinafter generally referred to as IRP-64) uses the hydrogen ion as the exchange ion. In an acidic aqueous medium, wherein the pH is less than about pH 4, the IRP-64 resin is in a nonionic state and exists as the free acid. When the acidic aqueous medium is at about pH 5, the IRP-64 resin's carboxyl groups liberate hydrogen ions and are converted in an acid-base reaction to the anionic form. The free hydrogen protonates a weakly basic active substance which then binds to the resin's acidic carboxylic acid group to form a water-insoluble complex. A quantity of the weakly basic active substance begins to bind onto the resin at pH 5 or greater. As the pH is made more basic, a greater quantity of the weakly basic active substance is solubilized and the equilibrium concentration of the weakly basic active substance is shifted to thus complex the solubilized active substance with the resin. Accordingly, an amount of the water-insoluble active substance is bound to the resin and a sufficiently low solution concentration of the water-insoluble active substance remaining in the suspension is maintained so as to effectively taste-mask the dosage form. Accordingly, for a given dosage strength, the term “substantially complexed” refers to a ratio of the amount of the active substance to the amount of resin available for complexing whereby the loading capacity of the resin is not exceeded.

Maintenance of a sufficiently low solution concentration of the water-insoluble active substance to effectively taste-mask the dosage form is also a function of the aqueous medium pH. For an aqueous medium at an initial pH, increasing the ratio of resin to active substance yields an inversely proportional solution concentration of the active substance because more resin is available to adsorb the active substance from solution. However, a proportional decrease in the aqueous medium pH (more acidic) also occurs. The aqueous medium pH decreases because more hydrogen ions are in solution as a result of an increased amount of available resin, resulting in protonation of more of the active substance. Accordingly, the solution concentration of the active substance decreases. Therefore, shifting either the aqueous medium pH or the ratio of resin to active substance, the equilibirum concentration is shifted to either favor complexing the active substance with the resin or maintaining the active substance in solution. The pH effects of an aqueous medium and ingredients therein are regulated in embodiments of the present invention by use of a neutralizing agent.

In embodiments of the present invention, the weakly acidic ion-exchange resin is Amberlite Resin Grade IRP-64 and the weakly basic active substance is risperidone. An embodiment of the invention includes a taste-masking effective amount of risperidone bound to the IRP-64 resin, leaving an amount of free risperidone in solution that is below a taste threshold concentration. In the context of the present invention, the term “taste threshold” refers to the inability to taste the bitterness associated with risperidone and the slight acidic taste and mouth feel of IRP-64 resin or the level at which these tastes are acceptable. For example, an aqueous medium at an initial pH of about 7 and a risperidone:resin ratio of greater than 1:4 yielded a free risperidone level of about 295 μg/mL remaining in solution, the taste of which was unacceptable. In another example, an aqueous medium at an initial pH of about 7 and a risperidone:resin ratio of 1:8 yielded a free risperidone level of about 2 μg/mL remaining in solution, the taste of which was acceptable.

Embodiments of the invention include an amount of free risperidone of less than about 25 μg/mL in solution, below the taste threshold concentration. Embodiments also include an amount of free risperidone selected from an amount of less than about 20 μg/mL, an amount of less than about 15 μg/mL, an amount of less than about 10 μg/mL, an amount of less than about 5 μg/mL, an amount of less than about 1 μg/mL or an amount of less than about 0.1 μg/mL.

Embodiments further include an amount of free risperidone selected from an amount in a range of from about 0.1 μg/mL to about 25 μg/mL, an amount in a range of from about 1 μg/mL to about 25 μg/mL, an amount in a range of from about 1 μg/mL to about 20 μg/mL, an amount in a range of from about 1 μg/mL to about 15 μg/mL, an amount in a range of from about 1 μg/mL to about 10 μg/mL, an amount in a range of from about 1 μg/mL to about 5 μg/mL, an amount in a range of from about 5 μg/mL to about 20 μg/mL, an amount in a range of from about 5 μg/mL to about 15 μg/mL, an amount in a range of from about 5 μg/mL to about 10 μg/mL or an amount in a range of about 10 μg/mL.

Embodiments of the invention include a final aqueous medium pH selected from a pH in a range of from about pH 5 to about pH 10, a pH in a range of from about pH 5 to about pH 7, a pH in a range of from about pH 5 to about pH 6, a pH in a range of from about pH 6 to about pH 7, a pH in a range from about 5.2 to about pH 6.6, a pH in a range of from about pH 5.4 to about pH 6.4, or a pH in a range of from about pH 5.6 to about pH 6.

Embodiments of the invention include a risperidone:IRP-64 resin ratio selected from a ratio in a range of from about 1:4 to about 1:12, a ratio in a range of from about 1:4 to about 1:10, a ratio in a range of from about 1:5 to about 1:10, a ratio in a range of from about 1:6 to about 1:10, a ratio in a range of from about 1:5 to about 1:8, a ratio in a range of from about 1:6 to about 1:8, a ratio in a range of from about 1:5 to about 1:7, a ratio in a range of from about 1:6 to about 1:7, a ratio in a range of from about 1:5 to about 1:6, or a ratio of about 1:6.

Aqueous medium ingredients include, but are not limited to, water, water miscible and immiscible solvents, carrier materials, surface stabilizers, structure-imparting water-soluble excipients, thickening agents, filling agents, lubricating agents, suspending agents, neutralizing agents, sweetening agents, flavoring agents, coloring agents, anti-foaming agents, preservatives, diluents, effervescent agents and the like or mixtures thereof.

Examples of solvents include, but are not limited to, dichloromethane, benzyl alcohol, tetrahydrofuran, methanol, toluene, dimethylformamide, 2-butanone, ethanol, propylene glycol, acetone, dimethyl sulfoxide, ethyl acetate, polyethylene glycol 400 and the like. In an embodiment of the present invention, a preferred water miscible solvent is ethanol.

The resinate may be contained in a carrier material, which forms a porous network or matrix. The carrier may be any water-soluble or water-dispersible material that is pharmaceutically acceptable, inert to the water-insoluble active substance, inert to the resinate complex and capable of forming a rapidly disintegrating network (i.e., disintegrates within a target time or less in the mouth; e.g., 10 seconds). Examples of substances that may be used as carrier materials include, but are not limited to, pharmaceutical grade gelatin, glycine, mannitol, hydrolyzed dextrose, dextran, dextrin, maltodextrin, alginates, hydroxyethyl cellulose, sodium carboxymethyl cellulose, microcrystalline cellulose, corn-syrup solids, pectin, carrageenan, agar, chitosan, locust bean gum, xanthan gum, guar gum, acacia gum, tragacanth, conjac flower, rice flower, wheat gluten, sodium starch glycolate, soy fiber protein, potato protein, papain or horseradish peroxidase.

Embodiments of the present invention include a carrier material selected from pharmaceutical grade gelatin, glycine, mannitol or mixtures thereof. Carrier materials may also be used as functional ingredients for other purposes in the present invention whereby such carrier materials (such as glycine and the like) may optionally be used to reduce the slight acidic mouth feel of IRP-64 resin to an acceptable level. Embodiments of the present invention include an amount of carrier material present in the aqueous medium in a range of at least from about 0.1% to about 5% by weight volume. In embodiments of the present invention, the acidic nature of gelatin caused a decrease in the aqueous medium pH, increased the solubility of free risperidone and competed with the resin for risperidone. However, the effect of the gelatin was neutralized by the addition of a neutralizing agent such as sodium hydroxide.

Useful surface stabilizers physically adhere to the surface of the active substance but do not chemically bond to or interact with the active substance. The surface stabilizer is adsorbed on the surface of the active substance while the individually adsorbed molecules of the surface stabilizer are essentially free of intermolecular cross-linkages. Accordingly, one or more surface stabilizers can be employed in a composition and method of the present invention. Examples of suitable surface stabilizers include, but are not limited to, various polymers, low molecular weight oligomers, natural products, and nonionic and ionic surfactants. Examples of surface stabilizers include gelatin, glycerine, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters [e.g., a commercially available Tween®: Tween 20®, Tween 80®, (trade names of ICI Specialty Chemicals) and the like]; a synthetic glycol polymer (such as propylene glycol, polypropylene glycol, polyethylene glycol, polyvinylpyrrolidone, polyvinylalcohol, polyoxyethylene copolymers, polyoxypropylene copolymers, polyethyleneoxide and the like: e.g., Carbowax 3550® or Carbowax 934® (trade names of Union Carbide)), polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetramethylbutyl)-phenol polymer with ethylene oxide and formaldehyde (also known as tyloxapol, superione, and triton), poloxamers (a block copolymer of ethylene oxide and propylene oxide)(e.g., Pluronics F68® and F108®); poloxamines (a tetrafunctional block copolymer derived from sequential addition of propylene oxide and ethylene oxide to ethylenediamine)(e.g., Tetronic 908®, also known as Poloxamine 908® or Tetronic 1508®, also known as T-1508, all trade names of the BASF Wyandotte Corporation); dialkylesters of sodium sulfosuccinic acid (e.g., a dioctyl ester of sodium sulfosuccinic acid)(Aerosol OT®, a trade name of American Cyanamid); a sodium lauryl sulfate (e.g., Duponol® P, a trade name of DuPont); an alkyl aryl polyether sulfonate (e.g., Tritons® X-200, a trade name of Rohm and Haas); a mixture of sucrose stearate and sucrose distearate (e.g., Crodestas® F-110 or Crodestas® SL-40, trade names of Croda Inc.) and the like. Most of these surface stabilizers are known pharmaceutical excipients and are described in detail in the Handbook of pharmaceutical Excipients, published jointly by the American Pharmaceutical Association and The Pharmaceutical Society of Great Britain (The Pharmaceutical Press, 1986).

Examples of structure-imparting water-soluble or water-dispersible excipients include, but are not limited to, a sugar (such as sucrose, lactose, glucose, mannose and the like), a sugar alcohol (such as mannitol, sorbitol, xylitol, erythritol, lactitol, maltitol and the like), a starch or modified starch (such as corn starch, potato starch, maize starch and the like), natural polymers or synthetic derivatives of a natural polymer (such as gelatin, xanthan gum, carrageenan, alginates, dextran, maltodextran and the like) and the like or mixtures thereof.

Thickening agents act to impart viscosity to the aqueous medium. Examples of thickening agents include, but are not limited to, natural gums (such as acacia, xanthan gum and the like), a high molecular weight cross-linked acrylic acid carbomer [such as Carbopol® 980, Carbopol® 974P (Carbomer 934P), Carbopol® 940 (all trade names of B.F. Goodrich & Co.)] and the like or mixtures thereof. The units of viscosity in the present invention are expressed in centipoises (cps) wherein the term “poise” refers to a unit of dynamic viscosity equal to the dynamic viscosity of a fluid in which there is a tangential force 1 dyne per square centimeter resisting the flow of two parallel fluid layers past each other when their differential velocity is 1 centimeter per second per centimeter of separation. In an embodiment of the present invention, the thickening agent is the carbomer Carbopol® 974P, the acidic nature of which causes a decrease in the aqueous medium pH, shifting the equilibrium to increase the solubility of free risperidone. However, the equilibrium shifting effect of the carbomer may be neutralized by the addition of a neutralizing agent such as sodium hydroxide.

Examples of filling agents include, but are not limited to, calcium disulfate, calcium trisulfate, calcium carbonate, microcrystalline cellulose, lactose monohydrate, lactose anhydrous, sucrose, mannitol, sorbitol, various starches and modified starches and the like or mixtures thereof.

Lubricating agents act on the ability of the active substance and resin powders to flow. Examples of lubricants include, but are not limited to, colloidal silicon dioxide (such as Aerosil® 200), talc, stearic acid, magnesium stearate, calcium stearate or silica gel.

Suspending agents act on the ability of the resinate to remain distributed in a suspension and thus maintain content uniformity of the active substance in suspension. Examples of suspending agents include, but are not limited to, propylene glycol, polyethylene glycol, glycerin and the like or mixtures thereof.

Neutralizing agents in the context of the present invention shift the equilibrium concentration of a solubilized weakly basic active substance and drive the active substance to favor complexing with a weakly acidic ion-exchange resin. The equilibrium concentration is shifted since a neutralizing agent is used to remove excess solubilized hydrogen ions present in the suspension as a result of using various acidic components (such as an acidic resin, an acidic carrier material, an acidic thickening agent and the like). Examples of neutralizing agents include, but are not limited to, sodium hydroxide.

Sweetening agents, flavoring agents and mixtures thereof used in the present invention are selected from those which are pharmaceutically acceptable, compatible with the attributes of an oral dosage formulation and adequately mask the slight acidic taste of IRP64 resin to below the taste threshold. Examples of sweetening agents include any natural or artificial sweetener (such as glucose, dextrose or fructose and the like or mixtures thereof, when not used as a carrier; saccharin and its various salts, cyclamate, aspartame, acesulfame-K and its sodium and calcium salts and the like or mixtures thereof; sucrose or sucralose; sugar alcohols such as sorbitol, mannitol, xylitol and the like or mixtures thereof) and the like or mixtures thereof.

Examples of flavoring agents include any natural or synthetic flavoring liquid (such as volatile oils, synthetic flavor oils, flavoring aromatics, oils, liquids, oleoresins and extracts derived from plants, leaves, flowers, fruits, stems and combinations thereof, including, but not limited to, spearmint, peppermint, lemon, orange, grape, lime or grapefruit citric oils or apple, pear, peach, grape, strawberry, raspberry, cherry, plum, pineapple, apricot, or other mint or fruit flavor essences), an aldehyde or ester (such as benzaldehyde (cherry, almond), citral, a-citral (lemon, lime), neral, beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and the like or mixtures thereof.

Examples of coloring agents include any pharmaceutically acceptable natural or synthetic dyes (such as Red 30 ferric oxide and the like) and the like or mixtures thereof.

Examples of anti-foaming agents include, but are not limited to, simethicone and the like or mixtures thereof.

Examples of preservatives include, but are not limited to, potassium sorbate, methylparaben, propylparaben, benzoic acid and its salts, other esters of parahydroxybenzoic acid (such as butylparaben, alcohols such as ethyl or benzyl alcohol), phenolic compounds (such as phenol) or quartemary compounds (such as benzalkonium chloride).

Examples of diluents include, but are not limited to, microcrystalline cellulose (such as Avicel® PH101 and Avicel® PH102), lactose (such as lactose monohydrate, lactose anhydrous and Pharmatose® DCL21), dibasic calcium phosphate (such as DCL21) or saccharides (such as mannitol, starch, sorbitol, sucrose and glucose) or mixtures thereof.

Examples of effervescent agents include effervescent combinations of an organic acid and a carbonate or bicarbonate. “Effervescent” refers to those agents which evolve gas. The gas-or bubble-generating action is often the result of the reaction of a soluble acid source and a carbonate source. The reaction of these two general classes of compounds produces carbon dioxide gas upon contact with water in saliva. Useful acids include: citric, tartaric, malic, fumaric, adipic, succinic and the like and acid salts and anhydrides thereof. Acid salts may also include sodium dihydrogen phosphate, disodium dihydrogen pyrophosphate, acid citrate salts, sodium acid sulfite and the like. While the food acids can be those indicated above, acid anhydrides of the above-described acids may also be used. Carbonate sources include dry solid carbonate and bicarbonate salts such as sodium bicarbonate, sodium carbonate, potassium bicarbonate, potassium carbonate, magnesium carbonate, sodium sesquicarbonate, sodium glycine carbonate, L-lysine carbonate, arginine carbonate, amorphous calcium carbonate and the like or mixtures thereof.

In an embodiment of the rapid-disintegrating, taste-masked oral dosage form of the present invention, the aqueous medium ingredients include, but are not limited to, water, carrier materials, structure-imparting water-soluble excipients, thickening agents, neutralizing agents, sweetening agents, flavoring agents, coloring agents, anti-foaming agents and the like or mixtures thereof.

Embodiments of the present invention include aqueous medium ingredients selected from water, a carrier material (such as gelatin and the like), structure-imparting water-soluble excipients (such as glycine, mannitol and the like or mixtures thereof), thickening agents (such as a carbomer or natural gum and the like or mixtures thereof), neutralizing agents (such as sodium hydroxide and the like), sweetening agents (such as aspartame, sucralose and the like or mixtures thereof), flavoring agents (such as peppermint oil and the like), coloring agents (such as ferric oxide and the like), anti-foaming agents (such as simethicone and the like) and the like or mixtures thereof.

In a preferred embodiment of the present invention, the carrier material is gelatin, the structure-imparting water-soluble excipients are glycine and mannitol, the thickening agent is a carbomer such as Carbopol® 974P or a natural gum such as xanthan gum, the neutralizing agent is sodium hydroxide, the sweetening agents are aspartame or sucralose or mixtures thereof, the flavoring agent is peppermint oil, the coloring agent is ferric oxide and the anti-foaming agent is simethicone.

Embodiments of the invention include ingredients which are present in a certain amount, wherein the ingredient and range of amount in milligrams (mg) per tablet is selected from:

| | Range |
|---|---|
| Ingredient | (mg per tablet) |
| Risperidone | 0.1-10 |
| Amberlite ® Resin IRP- | 1-50 |
| 64 | |
| Mannitol, USP | 1.5-20 |
| Gelatin, NF | 1.5-15 |
| Glycine, USP | 1-15 |
| Simethicone, USP | 0.007-0.06 |
| Carbopol 974P ®, NF | 0.03-0.5 |
| Sodium Hydroxide, NF | 0.01-0.2 |
| Pellets | |
| Aspartame, NF | 0.02-1.5 |
| Red 30 Ferric Oxide, NF | 0.002-0.2 |
| Peppermint Oil, NF | 0.05-0.8 |
| (Redistilled) | |
| Xanthan gum, NF | 0.02-0.8 |

Embodiments of the invention include ingredients which are present in a certain amount, wherein the ingredient and corresponding amount range in percent weight per weight of the dosage strength is selected from:

| Ingredient | Range (% w/w) |
|---|---|
| Risperidone | 0.01-1 |
| Amberlite ® Resin IRP-64 | 0.1-5 |
| Gelatin, NF | 0.15-1.5 |
| Mannitol, USP | 0.15-2 |
| Glycine, USP | 0.1-1.5 |
| Simethicone, USP | 0.0007-0.006 |
| Carbopol 974P ®, NF | 0.003-0.05 |
| Sodium Hydroxide, NF Pellets | 0.001-0.02 |
| Aspartame, NF | 0.002-0.15 |
| Red 30 Ferric Oxide, NF | 0.0002-0.02 |
| Peppermint Oil, NF (Redistilled) | 0.005-0.08 |
| Xanthan Gum, NF | 0.002-0.08 |
| Purified Water, USP | 88.594-99.47 |

Particular embodiments of the invention include ingredients which are present in a certain amount, wherein the ingredient and corresponding amount in milligrams (mg) per tablet is selected from:

| Ingredient | 0.5 mg | 1 mg | 2 mg |
|---|---|---|---|
| Risperidone | 0.5 | 1.0 | 2.0 |
| Amberlite ® Resin IRP-64 | 3.0 | 6.0 | 12.0 |
| Gelatin, NF | 3.25 | 6.5 | 13.0 |
| Mannitol, USP | 4.0 | 8.0 | 16.0 |
| Glycine, USP | 3.0 | 6.0 | 12.0 |
| Simethicone, USP | 0.01 | 0.02 | 0.04 |
| Carbopol 974P ®, NF | 0.075 | 0.15 | 0.30 |
| Sodium Hydroxide, NF Pellets | 0.039 | 0.078 | 0.156 |
| Aspartame, NF | 0.25 | 0.5 | 1.0 |
| Red 30 Ferric Oxide, NF | 0.003 | 0.00625 | 0.0125 |
| Peppermint Oil, NF (Redistilled) | 0.15 | 0.30 | 0.60 |
| Total (mg) | 14.3 | 28.6 | 57.1 |

Preferred aqueous medium ingredient amounts for embodiments of the present invention are those amounts wherein the pre-loading pH of the aqueous medium (i.e. prior to adding the risperidone and the IRP-64 resin) has been adjusted (made more basic) to maintain a solubilized risperidone concentration below the taste-threshold concentration after the resinate is formed.

For example, to maintain a final aqueous suspension pH in a range of from about pH 5 to about pH 7 and, thereby, a solubilized risperidone concentration of less than 10 μg/mL, the pre-loading pH of the aqueous medium is in a range from about at least pH 6 to about pH 8.

In a rapid-disintegrating, taste-masked oral dosage form of the present invention, aqueous medium ingredients (such as gelatin, carbomer and the like or mixtures thereof) were identified as having an influence on pre-loading pH of the aqueous medium and the rate at which the equilibrium concentration of the resin and active substance were reached. The addition of an optional neutralizing agent (such as sodium hydroxide) regulates effects which such ingredients have on the final suspension pH and subsequent free risperidone concentration.

### Manufacturing Process

The present invention provides a method for preparing a taste-masked resinate oral dosage form comprising the steps of:
forming a taste-masked resinate in an aqueous medium, wherein the resinate is a therapeutically effective amount of a water-insoluble, bitter-tasting pharmaceutically active substance complexed with an ion-exchange resin, wherein the aqueous medium comprises water and, optionally, one or more of the following: neutralizing agents, sweetening agents, flavoring agents, coloring agents, anti-foaming agents and the like or mixtures thereof, wherein the water-insoluble active substance is present in a water-insoluble base form and is complexed with the resin in a taste-masking effective amount, wherein the ion-exchange resin is a cationic ion-exchange resin;
forming a plurality of resinate dosage units; and
removing the water from the dosage units by drying by, for example, lyophilization, centrifugation, spray-drying, evaporation with or without a heat source, filtration with or without a vacuum or combinations thereof.

The method further comprises maintaining the active substance complexed to the ion-exchange resin in a taste-masking effective amount by adjusting the amount of the active substance and the amount of the ion-exchange resin to achieve a specified ratio. The active substance may be a water-insoluble base form of a water-insoluble active substance and the ion-exchange resin may be a cationic ion-exchange resin.

An embodiment of the method includes forming a taste-masked resinate in an aqueous medium wherein the medium is selected from water or an aqueous solution, wherein the solution optionally includes one or more of the following; carrier materials, structure-imparting water-soluble excipients, thickening agents, neutralizing agents, sweetening agents, flavoring agents, coloring agents, anti-foaming agents and the like or mixtures thereof.

Embodiments of such a method include optionally balancing the pH of the medium by using a neutralizing agent, whereby the active substance remains complexed with the ion-exchange resin in a taste-masking effective amount.

In such embodiments, the medium is removed from the taste-masked resinate by drying (such as by lyophization, centrifugation, spray-drying, evaporation with or without a heat source, filtration with or without a vacuum and the like or a combination thereof) to form a dried resinate, wherein the resinate is subsequently used in a dosage form selected form a rapid-disintegrating tablet, a rapid-disintegrating film, an effervescent tablet, a chewable tablet, a chewing gum, a suspension s sprinkle granule, a powder for reconstitution in a suspension or mixtures thereof.

Another method of the invention includes preparing a rapid-disintegrating, taste-masked tablet comprising the steps of:
forming a taste-masked resinate in an aqueous medium, wherein the taste-masked resinate is a therapeutically effective amount of a water-insoluble base form of a bitter-tasting, water-insoluble pharmaceutically active substance complexed in a taste-masking effective amount with a cationic ion-exchange resin, wherein the medium is a solution or suspension comprising water and optionally one or more of the following: carrier materials, structure-imparting water-soluble excipients, thickening agents, neutralizing agents, sweetening agents, flavoring agents, coloring agents, anti-foaming agents and the like or mixtures thereof;
if necessary, balancing the medium pH by adding an optional neutralizing agent, whereby an equilibrium concentration of the resin and the active substance is shifted to favor complexing the active substance with the resin, whereby a taste-masking effective amount of the active substance is maintained;
imparting viscosity to the medium by, for example, adding a thickening agent to the medium or homogenizing the medium, wherein the thickening agent is selected from a carbomer, a natural gum or mixtures thereof;
forming a plurality of rapid-disintegrating, taste-masked tablet dosage units; and
removing the water from the dosage units by drying by, for example, lyophilization, centrifugation, spray-drying, evaporation with or without a heat source filtrtion with or without a vacuum or combinations thereof.

The method includes adjusting the amount of the active substance and the amount of the ion-exchange resin to achieve a ratio whereby the active substance is complexed with the ion-exchange resin in a taste-masking effective amount. The active substance may be a base form of the water-insoluble active substance and the ion-exchange resin may be a cationic ion-exchange resin.

Preparing a Rapid-Disintegrating Taste-Masked Oral Dosage Form

The method of the invention may include the steps of mixing the ingredients to form a resinate in a suspension, homogenizing the suspension or adding thickening agent to the suspension to provide suspension viscosity, filling a plurality of divided dosage molds with the suspension, then freezing and lyophilizing the suspension in the molds to form a rapid-disintegrating, taste-masked tablet.

The mixing step may further include:

(1) charging a first vessel with an appropriate weight of purified water while the first vessel is under a vacuum and heating the water to a temperature in a range of from about 58° to about 62° C.;
(2) adding a first amount of simethicone to the first vessel and mixing for about 5 minutes;
(3) adding mannitol, glycine, aspartame and ferric oxide to the first vessel and mixing for about 5 minutes;
(4) adding risperidone and IRP-64 to the first vessel and mixing for about 5 minutes;
(5) releasing the vacuum from the first vessel and mixing for about 55 minutes;
(6) cooling the first vessel from a temperature in the range of from about 58° to about 62° C. to a temperature in a range of from about 37° to about 40° C.;
(7) adding a second amount of simethicone and gelatin to the first vessel and mixing for about 14 minutes at a suspension temperature in the range of from about 37° to about 40° C.;
(8) replacing vacuum and mixing for about 10 minutes;
(9) optionally adding carbomer and mixing for about 5 minutes;
(10) optionally adding xanthan gum to the suspension to obtain a suspension viscosity in a range of not less than 90 cps;
(11) adding sodium hydroxide and mixing for about 5 minutes;
(12) cooling the first vessel to a temperature in a range of from about 20° to about 25° C.;
(13) adding peppermint oil and mixing the suspension at a temperature of about 22° C.;
(14) releasing the vacuum after any foam remaining in the first vessel is eliminated.
   The homogenization step may further include:
(1) discharging the suspension from the first vessel to a second vessel and cooling the suspension to a temperature in a range of below about 28° C., a temperature in a range of about 180 to about 25° C., a temperature in a range of about 200 to about 25° C., or a temperature in a range of about 21° to about 23° C.;
(2) recirculating and mixing the contents of the second vessel;
(3) homogenizing the suspension in a second vessel at a pressure in a range of from about 4,000 psig to about 10,000 psig, a pressure in a range of from about 4,000 psig to about 8,000 psig, a pressure in a range of from about 4,000 psig to about 7,000 psig, a pressure in a range of from about 4,000 psig to about 6,000 psig, a pressure in a range of from about 5,000 psig to about 10,000 psig, a pressure in a range of from about 5,000 psig to about 8,000 psig, a pressure in a range of from about 5,000 psig to about 7,000 psig, a pressure in a range of from about 5,000 psig to about 6,000 psig, or a pressure in a range of about 5,000 psig;
(4) lowering the second vessel pressure to atmospheric pressure; and,
(5) mixing and recirculating the suspension for about 1 hour or until suspension viscosity is in a range of not less than 90 cps, a viscosity in a range of from about 90 cps to about 2500 cps, a viscosity in a range of from about 90 cps to about 1500 cps, a viscosity in a range of from about 90 cps to about 500 cps, a viscosity in a range of from about 90 cps to about 250 cps, a viscosity in a range of from about 90 cps to about 200 cps or a viscosity in a range of from about 90 cps to about 150 cps.
   The filling, freezing and lyophilization steps may further include:
(1) discharging the suspension from the second vessel to a filler system;
(2) filling a plurality of divided dosage molds with the suspension;
(3) freezing the suspension in the molds via a freeze tunnel;
(4) storing the molds at a temperature in a range of about −20° C.; and
(5) lyophilizing the suspension in the molds at a temperature in a range of about 40° C.

A preferred method of the invention comprises the steps of mixing the ingredients to form a resinate in a suspension, filling a plurality of divided dosage units with the suspension, drying the suspension in the dosage units to form a plurality of resinate dosage units, wherein the drying may be by, for example, lyophilization, centrifugation, spray-drying, evaporation with or without a heat source, filtration with or without a vacuum or combinations thereof, and wherein the resinate dosage units are subsequently used as a taste-masked active ingredient in a dosage form selected from a rapid-disintegrating tablet, a rapid-disintegrating film, an effervescent tablet, a chewable tablet, a chewing gum, a suspension, a sprinkle granule, a powder for reconstitution in a suspension or mixtures thereof.

The mixing step of the preferred method may further include:

(1) charging a first vessel with an appropriate weight of purified water while the first vessel is under a vacuum and heating the water to a temperature in a range of from about 58 to about 62° C.;
(2) adding a first amount of simethicone to the first vessel and mixing for about 5 minutes;
(3) adding risperidone and IRP-64 to the first vessel and mixing for about 5 minutes;
(4) releasing the vacuum from the first vessel and mixing for about 55 minutes; and
(5) cooling the first vessel to a temperature of below about 28° C., a temperature in a range of about 18 to about 25° C., a temperature in a range of from about 20 to about 25° C. or a temperature in a range of about 21 to about 23° C.

The rate of risperidone binding to the IRP-64 resin in the mixing step increased as a function of mixing temperature. In embodiments of the invention, the resinate is formed at a mixing temperature in a range of from about 25° C. to about 100° C., a mixing temperature in a range of from about 45° C. to about 80° C., a mixing temperature in a range of from about 60° C. to about 80° C., a mixing temperature in a range of from about 45° C. to about 65° C., a mixing temperature in a range of from about 58° C. to about 62° C. or a mixing temperature of about 60° C. Embodiments of the invention include a time period for mixing after adding the risperidone and IRP-64 resin to the first vessel of from about 15 minutes to about 24 hours or for a time period of about 1 hour, wherein the resinate suspension was formed.

The resinate suspension was then cooled to about 40° C. to prevent hydrolysis of the carrier material and the carrier material, thickening agent(s) and neutralizing agent were added.

Viscosity is imparted to the resinate suspension by the addition of a first thickening agent such as a carbomer, the addition of a second optional thickening agent such as a natural gum or by a step of homogenizing the suspension to obtain an optimum viscosity.

During the mixing step, the pH of the aqueous medium was lowered by the addition of various acidic ingredients (in particular, the weakly acidic IRP-64, the acidic gelatin, the acidic carbomer and the like), the addition of an optional neutralizing agent balanced the aqueous medium pH whereby the equilibirum concentration of risperidone and resin was shifted to form the resinate complex.

During the mixing step, neither the particle size of the resin nor the active substance affected the final solution concentration of the active substance or the ion-exchange capacity of the resin. The effect of resin particle size was limited to binding kinetics during the mixing step and content uniformity during the filling step. The effect of a smaller resin particle size during the mixing step was to increase the rate at which the equilibrium concentration of the active substance in solution was reached. The effect of a smaller resin particle size during the filling step was to improve content uniformity of the suspension as it was dispensed into the molds.

The IRP-64 used in the present invention, which was available from Rohm and Haas, had a particle size specification wherein 15.0%-30.0% of the resin had a particle size of greater than 0.075 mm (75 microns) and 1% maximum of the resin had a particle size greater than 0.150 mm (150 microns). An average resin particle size of less than 75 microns was found to improve content uniformity as the suspension was filled into the dosage molds.

The risperidone used in the present invention consisted of at least 99% of the substance having a particle size of less than 100 μm; in particular, at least 98% of the substance had a particle size of less than 74 μm; more particularly, at least 95% of the substance had a particle size of less than 45 μm.

The homogenization step was used to uniformly disperse the flavoring and coloring agents and optionally to build suspension viscosity.

### FORMULATION EXAMPLES

Representative embodiments of the present invention are illustrated in the specific examples that follow. The examples are solely offered by way of illustration; the invention should not be construed as being limited by the materials and conditions expressed.

### Example 1

Risperidone Binding to IRP-64 Resin in Aqueous Media

In an embodiment of the invention, the method involved mixing risperidone with IRP-64 ion-exchange resin in a 1:8 ratio of the amount of risperidone to the amount of resin. The resin and risperidone were added to either water, citrate-phosphate buffer at pH 5.93 or a 1.7% gelatin solution (Table 1a). The initial concentration of risperidone in the slurries was 15 mg/mL. The control consisted of risperidone in each of the three aqueous media.

Each slurry was placed into centrifuge tubes, capped and mixed with a Burrell Wrist action shaker model 75 for 24 hours. After 24 hours, the slurry was centrifuged at a relative centrifugal force of 38,724 g for 18 minutes. The supernatant was collected, filtered through a 0.45 μl nylon filter and assayed for risperidone by an isocratic HPLC method. The concentration of drug present in the supernatant represented the amount of unbound or “free” risperidone. The risperidone (control) concentrations presented in (Table 1 a) represent the solubility of risperidone drug substance in each of the specified media.

TABLE 1A
Concentration of Risperidone (μg/mL) in Supernatant
after 24 Hours Mixing

| | | | |
|---|---|---|---|
| | Purified | Citrate-Phosphate | |
| Media | Water | Buffer (pH 5.93) | 1.7% Gelatin |

| | | | |
|---|---|---|---|
| Risperidone:IRP-64 (1:6) | ˜3 | — | — |
| Risperidone:IRP-64 (1:8) | 6.35 | 41.69 | 7.45 |
| Risperidone (Control) | 85.82 | 4603.6 | 1062.1 |

Results

In each of the media evaluated, the resin significantly complexed with the risperidone. The total binding in the citrate-phosphate buffer was slightly lower due, most likely, to the presence of competing ions in the slurry.

### Example 2

Effect of Media pH on Free Risperidone and Taste-Masking

Taste studies indicated that no taste was associated with aqueous free risperidone concentrations of less than 25 μg/mL. This value was the indicator for effectiveness of taste-masking.

Risperidone was complexed with the IRP-64 resin in an aqueous media having with a basic pH (Table 2a). Compared to vehicle, the aqueous media pH was decreased by loading the media with a gelatin carrier agent and a carbomer thickening agent. The effect on media pH compared to vehicle media by the addition of gelatin and a carbomer thickening agent (0.04% solution of sodium CARBOPOL® 974P) is shown in (Table 2a). The sodium-carbomer solution was prepared by neutralizing a 5% (w/w) CARBOPOL® 974P solution with 5.34% (w/w) of a 5% (w/w) sodium hydroxide solution.

After adding risperidone and IRP-64 resin to the loaded aqueous media, the concentration of free risperidone in solution was assessed. A 10 mL sample of the suspension was centrifuged for 20 minutes. After centrifugation, the supernatant was filtered through a 0.45μ filter and assayed for risperidone using an isocratic HPLC method.

TABLE 2A
Effect of Loaded Media pH on Free Risperidone Concentration

| | | Final | Free |
|---|---|---|---|
| | Media | Suspension | Risperidone |
| Gelatin (%) | (pH) | (pH) | (μg/mL) |
| vehicle | 7.7 | 7.7 | 59 |
| 1.30% | 7.06 | 5.81 | 7.90 |
| 1.30% | 7.06 | 5.86 | 5.86 |

Results

The decrease in final suspension pH was due to loading with gelatin and carbomer and the hydrogen ions released by the resin as it complexed with the risperidone. An initial media pH in a range of from about 6.8 to about 7.1 yielded a final suspension pH in a range of from about 5.8 to about 5.9. A final suspension pH in such a range achieved a free risperidone concentration of less than 8 μg/mL in the final suspension.

### Example 3

Carbomer Effect on Media pH

The effect on media pH of changing the amount of carbomer (sodium-carbomer solution) present in the media was assessed. The media also contained 1.3% gelatin. Risperidone and IRP-64 (1:6 ratio) were added and mixed for two hours. The suspension pH decreased due to release of hydrogen ions from the resin.

TABLE 3A
Effect of Sodium-Carbomer Solution on pH and
Free Risperidone Level

| | Sodium-Carbomer Solution (%) | | | | | |
|---|---|---|---|---|---|---|
| | 0% | 0.01% | 0.02% | 0.03% | 0.04% | 0.05% |
| Media pH | 5.3 | 5.5 | 6.5 | 6.8 | 7.0 | 7.2 |
| Suspension pH | 5.3 | 5.4 | 5.6 | 5.8 | 5.7 | 5.7 |
| Free Risperidone | 11.5 | 11.0 | 6.9 | 2.1 | 2.0 | 0.7 |
| (μg/mL) | | | | | | |

Results

As shown in (Table 3a), increasing the amount of thickening agent in the media increased media pH. The change in media pH was greatest in media containing≧0.03% sodium-carbomer solution, increasingly binding more risperidone as the resin became more anionic. For effective binding to occur, the resin requires a preloaded environment around pH 5-7. Therefore, an initial vehicle pH greater than 6, in combination with the hydrogen ions liberated from the resin as the risperidone was bound provided a final suspension pH which maintained a taste-masking effective amount of free risperidone in solution.

### Example 4

Effect of Risperidone:Resin Ratio

Free Risperidone Concentration (0.04% Sodium Carbomer Solution)

The optimum ratio of risperidone to IRP-64 resin needed for taste-masking was determined by testing various risperidone to resin ratios. The aqueous media contained 1.3% gelatin and a 0.04% sodium carbomer solution.

TABLE 4A
Effect of Ratio on Free Risperidone Concentration
(0.04% Sodium Carbomer Solution)

| | Risperidone:IRP64 | | | | | |
|---|---|---|---|---|---|---|
| | 1:3 | 1:4 | 1:6 | 1:6 | 1:6 | 1:8 |
| Media pH | 7.1 | 7.1 | 7.1 | 7.1 | 7.1 | 7.2 |
| Suspension pH | 6.7 | 6.4 | 6 | 5.8 | 5.9 | 5.7 |
| Free Risperidone (μg/mL) | 295.5 | 28.62 | 6.76 | 5.9 | 7.9 | 1.6 |

Results

As shown in (Table 4a), the media pH was in the desired pH range for drug loading and the suspension pH decreased as IRP64 concentration increased. The decrease was a result of resin proton release and the protonation of risperidone. The higher pH seen with the lower ratios (1:3 to 1:4) was most likely due to the presence of protonated risperidone. Accordingly, risperidone:resin ratios greater than 1:4 will have free risperidone concentrations less than 25 μg/mL. The difference seen in free risperidone concentrations between the 1:3 and 1:8 ratios was not solely due to a pH effect, but more due to the amount of resin present in the suspension. The 1:8 ratio had greater risperidone loading at a lower pH because there was more resin available to adsorb the risperidone in solution.

Free Risperidone Concentration (0.03% Sodium-Carbomer Solution)

The influence of pH and risperidone:IRP-64 ratio on free risperidone drug concentration was further investigated. The above experiment was repeated using a 0.03% sodium-carbomer solution. As shown in (Table 4b), over a pH range of 6.0 to 6.3, an increase in amount of resin decreased the amount of free risperidone.

TABLE 4B
Effect of Ratio on Free Risperidone Concentration
(0.03% Sodium-Carbomer Solution)

| | Risperidone:IRP64 | | |
|---|---|---|---|
| | 1:4 | 1:5 | 1:6 |
| Media pH | 6.9 | 6.9 | 6.9 |
| Suspension pH | 6.3 | 6.1 | 6.0 |
| Free Risperidone (μg/mL) | 43.1 | 10.8 | 4.8 |

Results

The free risperidone concentration presented in (Table 4b) illustrates that a ratio greater than 1:4 of risperidone:IRP64 provided adequate taste-masking.

### Example 5

Effect of Mixing Order

Media pH (0.04% Sodium-Carbomer Solution)

The effect on final suspension pH and, by implication, free risperidone concentration in an aqueous media containing 1.3% gelatin and 0.04% sodium-carbomer solution as a result of changing the order of mixing is shown in (Table 5a). The first ingredient was added to the media and mixed for one hour, then the second ingredient was added and the suspension was mixed for one hour. Accordingly, each suspension was mixed for a total of 2 hours.

TABLE 5A
Effect of Mixing Order on Media pH

| | Risperidone, | Powder Blend | IRP64, |
|---|---|---|---|
| Mixing Order | IRP-64 | (control) | Risperidone |
| Media pH | 7.42 | 7.42 | 5.21 |
| Final Suspension pH | 5.7 | 5.7 | 5.7 |

Results

Regardless of the order of addition, the pH of all three systems attained and reached a plateau at pH 5.7.

### Example 6

Effect of Resin Particle Size

Resin Binding

The effect of resin particle size on risperidone binding was evaluated for a 1:6 risperidone to resin ratio in media at 22° C. Three different particle size cuts from a single IRP64 resin lot were obtained by sieving. As shown in (Table 6a), the particle size cuts assessed at certain intervals while mixing were: 20-73 microns, 74-125 microns and >125 microns. A stir-bar was used for mixing.

TABLE 6A
Effect of Resin Particle Size on Free Risperidone Concentration*

| Particle Size | Free Risperidone (μg/mL) | | |
|---|---|---|---|
| (microns) | 2 h Mixing | 4.5 h Mixing | Overnight |
| 20-73 | 8.32 ± 0.16 | 5.83 ± 0.19 | 5.10 ± 0.15 |
| 74-125 | 12.59 ± 0.26 | 7.85 ± 0.25 | 4.51 ± 0.69 |
| >125 | 18.90 ± 0.15 | 9.61 ± 0.26 | 5.61 ± 0.13 |

*mean of n = 3; free risperidone ±1 standard deviation

Results

Resin particle size did not affect the equilibrium free risperidone level, but rather the kinetics of binding. The smaller the particle size, the faster the equilibrium free risperidone concentration was attained. After two hours, the free risperidone level was below the taste threshold level of 25 μg/mL for all size cuts.

### Example 7

Effect of Temperature on Binding

The effect of the process temperature on binding of risperidone:IRP64 at a 1:6 ratio was investigated in aqueous media at three different mixing temperatures: 22° C., 40° C. and 60° C. The aqueous media ingredients included a known volume of water, mannitol, glycine and ferric oxide maintained at 22° C., 40° C., and 60° C., respectively, mixed for one hour with overhead stirring. After one hour, each system was brought to 40° C. and 1.3% gelatin and 8% CARBOPOL gel were added. After the gelatin was dissolved, each system was cooled to 22° C. and mixed overnight prior to pH and free risperidone determination.

TABLE 7A
Effect of Temperature on Free Risperidone Concentration
Free Risperidone (μg/mL)¹

| | 3.5 hours | | 6 hours | | Overnight | |
|---|---|---|---|---|---|---|
| Temp | pH | (μg/mL) | pH | (μg/mL) | pH | (μg/mL) |
| 22° C. | 5.86 | 8.36 ± 0.08 | 5.83 | 8.75 ± 0.59 | 5.86 | 8.50 ± 0.07 |
| 40° C. | 5.86 | 8.52 ± 0.54 | 5.87 | 9.69 ± 0.52 | 5.89 | 9.82 ± 0.38 |
| 60° C. | 5.90 | 5.52 ± 0.13 | 5.90 | 7.04 ± 0.41 | 5.89 | 7.35 ± 0.29 |

¹Average of 3 samples ± 1 standard deviation

Results

As shown in (Table 7a), binding equilibrium was reached within 3.5 hours for the three temperature systems. There was no major change in the free risperidone level at 22° C. and 40° C. However, lower free risperidone levels observed at 60° C. demonstrated increased binding compared to the 22° C. and 40° C. systems.

Effect of Mixing Order on Binding

The effect of mixing order on binding of risperidone:IRP64 at a 1:6 ratio was investigated in aqueous media at a mixing temperature of 80° C. The aqueous media ingredients included a known volume of water, mannitol, glycine and ferric oxide maintained at 80° C. and mixed for one hour with overhead stirring. After one hour, the media was brought to 40° C. and 1.3% gelatin and 0.04% sodium-carbomer solution were added in various orders. After the gelatin was dissolved, each system was cooled to 22° C. and mixed overnight prior to pH and free risperidone determination.

TABLE 7B
Effect of Mixing Order

| Mixing Order | PH | Free Risperidone (μg/mL) |
|---|---|---|
| (1) IRP64, (2) risperidone, | 5.42 | 3.42 |
| (3) 0.04% solution, (4) | | |
| gelatin | | |
| (1) IRP64, (2) risperidone, | 5.44 | 1.94 |
| (3) gelatin, (4) 0.04% | | |
| solution | | |
| (1) 0.04% solution, (2) | 5.38 | 3.42 |
| IRP64, (3) risperidone, (4) | | |
| gelatin | | |
| (1) 0.04% solution, (2) | 5.42 | 7.47 |
| gelatin, (3) IRP64, (4) | | |
| risperidone | | |

Results

As shown in (Table 7b), at temperatures ≧60° C. (in this case, a risperidone:IRP-64 mixing temperature of 80° C.), the binding of risperidone to resin increased.

### Conclusion From Examples 1-7

The formation of a taste-masked risperidone resinate occurs when risperidone complexes with the IRP64 resin by ion-exchange as a result of an acid-base reaction. Dispersion of risperidone in water promoted the basic environment for binding to occur. Binding occurred more readily with a media at pH>4; conversely risperidone was increasingly freed as media pH was lowered. In a pH 5.5 media, hydrogen ions are liberated from carboxylic acid groups on resin molecules and risperidone is in a protonated form. The resinate forms when the protonated risperidone in solution binds to the acidic carboxylic acid group of the resin. The resulting risperidone-IRP64 complex is insoluble in water and, as a result, is taste-masked.

The free risperidone level of a (1:6) risperidone-IRP64 complex in water is approximately 3 μg/mL. As any aqueous medium ingredients are added, any changes in the medium pH can be countered by the addition of a neutralizing agent to bring the pH back to >4. The addition of a neutralizing agent such as sodium hydroxide minimizes the effects any aqueous medium ingredient had on the equilibrium concentration of risperidone by adjusting the final suspension pH to favor the binding of risperidone onto the resin.

Example 8

Taste Evaluation

Study Parameters

The taste evaluation study for a 0.5 mg strength of the risperidone rapid-disintegrating tablet was a single dose cross over trial in six volunteers. Taste evaluation occurred immediately before dosing, 5 minutes, 20 minutes, 40 minutes, 60 minutes, 90 minutes, 2 hours, 3 hours, 5 hours, 8 and 24 hours after intake of a single dose of the risperidone rapid-disintegrating tablet or the risperidone filmcoated tablet in each arm of the study. A wash-out period of at least one week occurred before administration of the second dosage form.

0.5 mg Strength Study Results

Taste results from the six volunteer subjects regarding the 0.5 mg risperidone rapid-disintegrating tablet were positive. Only one subject perceived a bitter taste 20 and 60 minutes after dosing. The intensity of bitterness was not mentioned. However, this same subject perceived a neutral taste at the 40 minute time point. This subject also qualitatively described the taste at five minutes as pleasant and at 20 minutes to 24 hours as neutral.

### CONCLUSION

Based on the combined study results, the bitter taste of risperidone in a rapid-disintegrating tablet was adequately taste-masked and was comparable to the filmcoated marketed tablet.

It is to be understood that the preceding description of the invention and its various embodiments has emphasized certain embodiments by way of example. Numerous other embodiments not specifically elaborated on or discussed may nevertheless fall within the spirit and scope of the present invention or the following claims and are intended to be included.

1. A taste-masked resinate comprising a water-insoluble active substance complexed in a taste-masking effective amount with an ion-exchange resin.

2. A dosage form comprising the taste-masked resinate of claim 1, the dosage form selected from the group consisting of a rapid-disintegrating tablet, a rapid-disintegrating film, an effervescent tablet, a chewable tablet, a chewing gum, a suspension, a sprinkle granule and a powder for reconstitution in a suspension.

3. The dosage form of claim 2 wherein the dosage form is selected from the group consisting of a rapid-disintegrating tablet and a suspension.

4. The taste-masked resinate of claim 1 comprising a base of a water-insoluble active substance and a cationic ion-exchange resin.

5. The taste-masked resinate of claim 1 comprising a water-insoluble active substance complexed to an ion-exchange resin in a ratio selected from the group consisting of a range from about 1:4 to about 1:12, a ratio in a range of from about 1:4 to about 1:10, a ratio in a range of from about 1:5 to about 1:10, a ratio in a range of from about 1:6 to about 1:10, a ratio in a range of from about 1:5 to about 1:8, a ratio in a range of from about 1:6 to about 1:8, a ratio in a range of from about 1:5 to about 1:7, a ratio in a range of from about 1:6 to about 1:7, a ratio in a range of from about 1:5 to about 1:6, or a ratio of about 1:6.

6. The taste-masked resinate of claim 5 comprising a water-insoluble active substance complexed to an ion-exchange resin in a ratio of between about 1:6 to about 1:8.

7. The taste-masked resinate of claim 6 comprising a water-insoluble active substance complexed to an ion-exchange resin in a ratio of about 1:6.

8. The taste-masked resinate of claim 1 wherein the water-insoluble active substance is complexed with the ion-exchange resin in a medium selected from the group consisting of water and a buffered aqueous solution.

9. The taste-masked resinate of claim 8 wherein a pH of the medium is controlled to a pH greater than about 4 by addition of a neutralizing agent.

10. A method for preparing a taste-masked resinate comprising allowing a specified amount of a water-insoluble active substance to complex to a specified amount of an ion-exchange resin.

11. The method of claim 10, wherein the water-insoluble active substance complexes to the ion-exchange resin in a ratio selected from the group consisting of a ratio in a range of from about 1:4 to about 1:12, a ratio in a range of from about 1:4 to about 1:10, a ratio in a range of from about 1:5 to about 1:10, a ratio in a range of from about 1:6 to about 1:10, a ratio in a range of from about 1:5 to about 1:8, a ratio in a range of from about 1:6 to about 1:8, a ratio in a range of from about 1:5 to about 1:7, a ratio in a range of from about 1:6 to about 1:7, a ratio in a range of from about 1:5 to about 1:6, or a ratio of about 1:6.

12. The method of claim 11, wherein the water-insoluble active substance complexes to the ion-exchange resin in a ratio of between about 1:6 to about 1:8.

13. The method of claim 12, wherein the water-insoluble active substance complexes to the ion-exchange resin in a ratio of about 1:6.

14. A pharmaceutical composition comprising a taste-masked resinate, wherein the taste-masked resinate comprises a therapeutically effective amount of a water-insoluble active substance complexed in a taste-masking effective amount with an ion-exchange resin.

15. A method for preparing a rapid-disintegrating, taste-masked dosage unit comprising:
forming the taste-masked resinate of claim 1 in an aqueous medium,
if necessary, imparting viscosity to the aqueous medium, wherein said viscosity can be imparted by adding a thickening or by homogenization; and
forming a plurality of dosage units; and
removing water from the dosage units by drying.
